(19) United States
(12) Patent Application Publication
(10) Pub. No.: US 2013/0255681 A1
(43) Pub. Date: Oct. 3, 2013

(54) DATA COLLECTION SYSTEM AND METHOD USING PARAMETRIC-BASED SAMPLING RATES

(71) Applicants: Richard Batch, Longmont, CO (US); Robert T. Boyer, Longmont, CO (US)

(72) Inventors: Richard Batch, Longmont, CO (US); Robert T. Boyer, Longmont, CO (US)

(73) Assignee: Nellcor Puritan Bennett LLC, Boulder, CO (US)

(21) Appl. No.: 13/435,398

(22) Filed: Mar. 30, 2012

(51) Int. Cl. A61B 5/0205; A61B 5/00; A61M 16/00

(52) U.S. Cl. 128/204.21; 600/300; 600/484

(57) ABSTRACT

The present disclosure relates to a system and method for sampling parametric data provided with a patient monitoring device. A disclosed method includes sampling parametric data associated with a patient and adjusting a sampling interval during the sampling of the parametric data based on a change in a frequency of a periodic physiological event associated with the patient. A disclosed system includes a server computer operative to sample parametric data associated with a patient and to adjust a sampling interval during the sampling of the parametric data based on a change in a frequency of a periodic physiological event associated with the patient.

### FIELD OF THE DISCLOSURE

The present disclosure relates generally to the field of patient monitoring systems, and more particularly to systems and methods for collecting data provided with one or more patient monitoring devices.

### BACKGROUND AND SUMMARY

A patient monitoring device measures parametric data associated with a patient that is connected to the device. Ventilators, for example, measure a large set of patient parameters including oxygen level, air pressure, air volume, air flow, and other parameters. Similarly, heart monitors measure various patient parameters associated with cardiac monitoring, such as, for example, pulse rate and other electrocardiogram (ECG) data. Some patient monitoring systems monitor patient parameters continuously. For example, ventilators may measure patient parameters on a breath-to-breath basis where the measured data is subject to change or updates with each breath of the patient. Other patient monitoring devices monitor patient parameters in a similar fashion. Heart monitors, for example, may collect a new set of parametric data with each pulse of the patient.

Hospitals and other patient care facilities often include one or more data collection servers that collect the parametric data monitored by the patient monitoring devices and other data, such as biometric, medical, and/or device configuration data. The collected data may be used for predictive analysis of a patient or group of patients or for diagnosing a patient's illness, for example. Patient monitoring devices typically report measured data upon request from the data collection system. As such, modern data collection systems collect the parametric data provided with the patient monitoring devices at a fixed or regular sampling rate. Oversampling the data by the data collection system negatively affects communication bandwidth and can lead to a failure or fault with the overworked patient monitoring device. As such, a data collection system may request data provided with a patient monitoring device at large sampling intervals, such as intervals of 30 seconds, one minute, two minutes, etc. Any data updates or events that occur between the sampling intervals are either lost or delayed until the next sampling interval occurs.

According to an illustrative embodiment of the present disclosure, a method of sampling parametric data by a data collection system is provided. The parametric data is provided with a ventilator system and is associated with a patient monitored by the ventilator system. The method includes receiving information associated with a first respiration rate of a monitored patient. The information associated with the first respiration rate is provided with the ventilator system. The method includes setting a data sampling rate to substantially match the first respiration rate, and sampling parametric data associated with the monitored patient at the data sampling rate. The parametric data is provided with the ventilator system. The method further includes receiving information associated with a second respiration rate of the monitored patient. The second respiration rate is different from the first respiration rate. The method further includes adjusting, during the sampling of the parametric data, the data sampling rate to substantially match the second respiration rate, and sampling parametric data associated with the monitored patient at the adjusted data sampling rate.

In one example, the method includes sending a request for the parametric data associated with the patient to the ventilator system. In another example, the method further includes receiving the requested parametric data and a respiration rate from the ventilator system upon sending the request. In yet another example, the method includes receiving a notification from the ventilator system following each of a plurality of successive respiration cycles of the monitored patient, the notification indicating that parametric data associated with the previous respiration cycle of the patient is available at the ventilator system. In still another example, the method includes sending a request to the ventilator system for the available parametric data associated with the previous respiration cycle of the patient following the receipt of each notification, and receiving the requested parametric data at the data collection system following the sending of each request. In another example, the method includes limiting the adjusted data sampling rate to a predetermined maximum rate upon the second respiration rate exceeding the predetermined maximum rate.

According to another illustrative embodiment of the present disclosure, a method of sampling data provided with a patient monitoring device is provided. The method includes sampling parametric data associated with a patient and provided with a patient monitoring device, determining a frequency of a periodic physiological event associated with the patient, and adjusting a sampling rate during the sampling of the parametric data based on a change in the frequency of the periodic physiological event associated with the patient. In one example, the method includes receiving information associated with the frequency of the periodic physiological event from the patient monitoring device. In another example, the method includes setting the sampling rate to match the frequency of the periodic physiological event associated with the patient. In yet another example, the method includes receiving a notification of each of a plurality of successive occurrences of the periodic physiological event associated with the patient. In still another example, the sampling rate is adjusted to sample the parametric data associated with the patient following the receipt of each notification. In another example, the periodic physiological event associated with the patient includes at least one of respiration and pulse.

According to yet another illustrative embodiment of the present disclosure, a data collection system is provided including a database and at least one server computer. The at least one server computer is operative to sample parametric data associated with a patient and to store the sampled parametric data in the database. The parametric data is provided with a patient monitoring device. The at least one server computer adjusts a sampling frequency during a sampling of the parametric data based on a change in a determined frequency of a periodic physiological event associated with the patient. In one example, the at least one server computer is operative to sample parametric data provided with each of a plurality of patient monitoring devices at a different sampling frequency. In another example, the at least one server computer receives information associated with the frequency of the periodic physiological event from the patient monitoring device.

### BRIEF DESCRIPTION OF THE DRAWINGS

The detailed description of the drawings particularly refers to the accompanying figures in which:

FIG. 1 is a block diagram of an exemplary data collection system according to one embodiment for collecting data from one or more patient monitoring devices;

FIG. 2 is an exemplary block diagram of the data collection system of FIG. 1 including data collection logic operative to sample data from a ventilator system at a variable sampling rate;

FIG. 3 is a flow chart of an exemplary method of operation of the data collection system of FIGS. 1 and 2;

FIG. 4 is a flow chart of another exemplary method of operation of the data collection system of FIGS. 1 and 2; and

FIG. 5 is a flow chart of another exemplary method of operation of the data collection system of FIGS. 1 and 2.

### DETAILED DESCRIPTION OF THE DRAWINGS

For the purposes of promoting an understanding of the principles of the present disclosure, reference will now be made to the embodiments illustrated in the drawings, which are described herein. The embodiments disclosed herein are not intended to be exhaustive or to limit the invention to the precise form disclosed. Rather, the embodiments are chosen and described so that others skilled in the art may utilize their teachings. Therefore, no limitation of the scope of the claimed invention is thereby intended. The present invention includes any alterations and further modifications of the illustrated devices and described methods and further applications of the principles of the invention which would normally occur to one skilled in the art to which the invention relates.

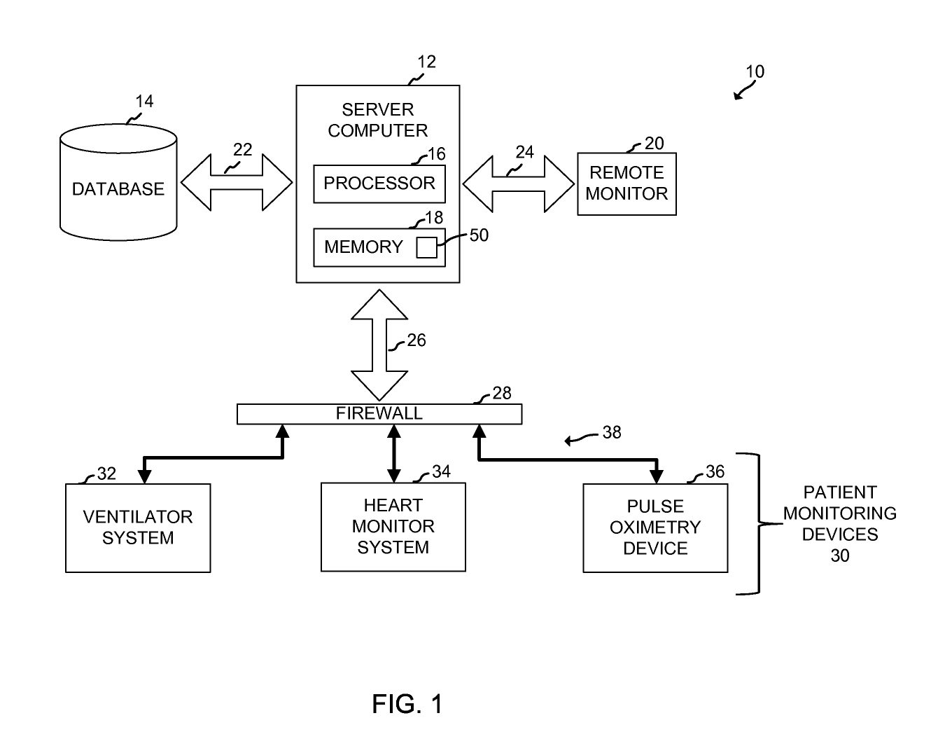

Referring to FIG. 1, an exemplary data collection system 10 is illustrated according to one embodiment. Data collection system 10 is operative to collect patient data provided with one or more patient monitoring devices 30. As described herein, data collection system 10 is configured to sample or poll data from each patient monitoring device 30 at dynamically adjustable sampling rates based on the detected physiological responses of monitored patients.

Data collection system 10 includes a server computer 12 and a server database 14 in communication with server computer 12. Server computer 12 includes a processor 16 and a memory 18 accessible by processor 16. Memory 18, which comprises one or more memory locations, includes software containing instructions executable by processor 16. Memory 18 illustratively includes data collection logic 50 comprising software or firmware code that, when executed by processor 16, causes server computer 12 to retrieve patient data from patient monitoring devices 30 at variable sampling rates, as described herein. Memory 18 may be internal or external to server computer 12. Processor 16 includes any suitable processing device or devices operative to execute the logic stored at memory 18. For example, processor 16 may include one or more programmable processors (e.g., central processing unit (CPU) devices), application-specific integrated circuits (ASICs), field-programmable gate arrays (FPGAs), digital signal processors (DSPs), hardwired logic, or combinations thereof. Server computer 12 is operative to route patient data received from patient monitoring devices 30 to server database 14 for storage.

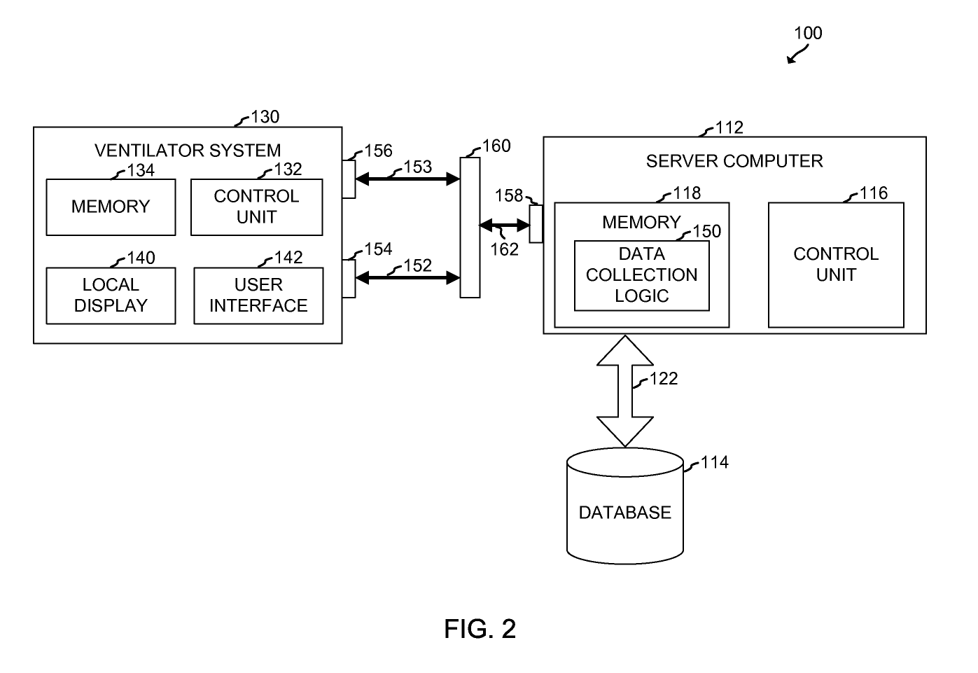

Server computer 12 receives various types of data from patient monitoring devices 30 and from other local and remote data sources. In one embodiment, devices 30 and/or other point-of-care devices in communication with server computer 12 route biometric, medical, and/or device configuration data entered by a clinician or device technician via a user interface (see, for example, user interface 142 of FIG. 2). Biometric data includes, for example, age, gender, health condition, skin pigmentation, nail polish color, and other information describing a patient. Medical data includes, for example, drugs, treatment history, and other relevant medical data of the patient. In addition, clinical event data may be entered by a patient or a medical professional, such as data related to drug administrations and arterial blood gas draws, for example. Further, device identification and capability data, device alarm information, research data from external facilities or applications, and other suitable data may be routed to server computer 12 and stored in database 14.

An optional remote monitor 20 is illustratively coupled to server computer 12 via a communication link 24, such as a computer networking protocol link (e.g., internet protocol (IP)). Remote monitor 20 includes any remote computer device or user interface operative to remotely communicate with server computer 12 and to access database 14. Database 14 is coupled to server computer 12 via a data bus 22, although computer 12 may alternatively communicate with database 14 over a computer networking protocol such as IP. Database 14 includes one or more data stores for storing the data provided with server computer 12. The stored data is accessible by a point of care application (e.g., a patient monitoring device 30) or by other local and remote applications (e.g., remote monitor 20) having authorized access to the database 14.

Patient monitoring devices 30 include point-of-care medical devices that are operative to measure one or more parameters of a patient and report the measured data to server computer 12 and/or to a local display. In the illustrated embodiment, patient monitoring devices 30 include a ventilator system 32, a heart monitor system 34, and a pulse oximetry device 36, although other suitable medical devices or systems may be provided that are operative to measure patient parameters. Exemplary parametric data monitored by devices 30 includes physiological data of the patient. Depending on the type of patient monitoring device 30, exemplary physiological data includes blood pressure, blood oxygen levels, air volume and pressure, ECG data, etc. Devices 30 also provide alarm data and data related to physiological events or responses associated with the patient. For example, ventilator 32 detects inhalation/exhalation events, and heart monitor 34 detects pulse events that are detected based on the monitored parametric data. Server computer 12 includes an encoder (not shown) operative to decode all received parametric data. Server computer 12 also normalizes the received and decoded parametric data in preparation for storage in database 14.

In one embodiment, patient monitoring devices 30 may store the monitored data in an internal or external local memory, as described herein with respect to FIG. 2. Upon receiving a data request from server computer 12, the patient monitoring device 30 provides the requested parametric data to server computer 12. Each patient monitoring device 32, 34, 36 is in communication with server computer 12 via communication links 26, 38 to transmit the data to server computer 12 and to receive the requests from server computer 12. In the illustrated embodiment of FIG. 1, devices 30 and server computer 12 communicate and transmit data over a computer networking protocol, such as an internet protocol (IP) format including Transmission Control Protocol/Internet Protocol (TCP/IP) or User Datagram Protocol (UDP), for example. In one embodiment, devices 30 and server computer 12 communicate using a wireless (e.g., Wi-Fi) network, an Ethernet network, or other local or wide area network (LAN or WAN). An optional communication firewall 28 is illustratively provided between server 12 and patient monitoring devices 30 to protect against unauthorized access of server computer 12. Server computer 12 may alternatively be coupled to a patient monitoring device 30 over a direct communication interface, such as via serial or universal serial bus (USB) communication, for example.

In another embodiment, data collection system 10 includes multiple server computers 12 networked together as a server farm. Such a server configuration may provide, for example, load balancing during data collection from multiple patient monitoring devices 30. A network of server computers 12 also may provide data replication capabilities such that data from devices 30 may be replicated and stored in multiple databases 14.

Referring to FIG. 2, another exemplary data collection system 100 is illustrated. Data collection system 100 of FIG. 2 may be viewed as one embodiment of the data collection system 10 of FIG. 1. Data collection 100 is illustratively coupled to a single patient monitoring device, i.e., ventilator system 130, but additional patient monitoring devices may coupled to data collection system 100. Data collection systems 10, 100 of FIGS. 1 and 2 each include respective data collection logic 50, 150 operative to manage and control data collection from patient monitoring device(s) 30, as described herein.

Data collection system 100 includes a computer 112 coupled to a ventilator system 130. Computer 112 may be any suitable computer device, such as a server, a laptop, a desktop, or a tablet computer, for example, operative to collect and route parametric data received from ventilator system 130 to a database 114. Computer 112 includes a control unit 116, such as a CPU or other suitable processing device(s), and a memory 118 accessible by the control unit 116. Memory 118 includes logic, such as software or firmware, containing instructions executable by control unit 116. Similar to data collection logic 50 of FIG. 1, memory 118 of FIG. 2 includes data collection logic 150 comprising control logic, such as software and/or firmware code, operative to control data collection from ventilator system 130 including setting and adjusting the data sampling rates, as described herein. The following illustrates an exemplary portion of program instructions used by data collection logic 150 (and data collection logic 50 of FIG. 1) to control the data sampling:

### Exemplary Program Instructions of Data Collection Logic 150

```
    /// <summary>
    /// Overrides the ProcessWriteQueue to check if it has been
one-breath's time since data was last requested.
    /// </summary>
    public override void ProcessWriteQueue( )
    {
      if ( mNextRequest <= DateTime.Now )
      {
        bool sendOwnMsg = true;
        mLastRequest = DateTime.Now ;
        // Determine next request time based on respiratory rate -
        account for round-off in rate.
        double addTime = 60.0 * (mRespiratoryRate − 0.5) /
( mRespiratoryRate * mRespiratoryRate ) ;
        if (addTime < MIN_UPDATE_REQUEST_TIME)
          addTime = MIN_UPDATE_REQUEST_TIME;
        mNextRequest = mLastRequest.AddSeconds( addTime );
        if ( sendOwnMsg )
        {
          if (string.Equals(PB840Message.SNDA, ReqMsg))
          {
            MessagesSent++;
            mOutputStream.write(mSndA, 0, mSndA.Length);
          }
          else
          {
            MessagesSent++;
            mOutputStream.write(mSndF, 0, mSndF.Length);
          }
        }
      }
    }
  }
    /// <summary>
    /// Pre-Process the received message for any service request messages
that might be pending. Also, process any message content
    /// data that directly affects the protocol handling, such as extracting
the respiratory rate to determine next request time.
    /// </summary>
    /// <param name=“objMessage”>Decoded ventilator
    message</param>
    /// <returns></returns>
    public override bool PreProcessForResponse( object objMessage )
    {
      bool retval = true ;  // in most all cases, we still want to attempt
to process the full message (even when invalid - gets picked-up elsewhere)
      if ( IsValidMessage( objMessage ) )
      {
              double respRate =
Convert.ToDouble(((PB840_MISCA)objMessage).RespiratoryRate);
              mRespiratoryRate = respRate;
      }
      return( retval ) ;
    }
```

Computer 112 is in communication with database 114 via communication link 122. In one embodiment, link 122 provides wireless or wired communication over an IP network, Ethernet network, or other suitable local or remote communication network. Ventilator system 130 includes a pair of communication ports 154, 156 for communicating with an external device, illustratively computer 112. In one embodiment, ports 154, 156 are serial communication ports 154, 156. Alternatively, ports 154, 156 may include universal serial bus (USB) ports 154, 156, although other types and numbers of ports 154, 156 may be provided. Communication cables 152, 153 are coupled to respective ports 154, 156 of ventilator system 130 and to a routing device 160. A communication link 162 is provided between routing device 160 and a communication port 158 of computer 112. In one embodiment, communication link 162 is a wired or wireless internet protocol (IP) link, such as TCP/IP or UDP. As such, routing device 160 is operative to convert serial or USB data from ventilator system 130 to an IP format for computer 112, and vice versa, for transferring the parametric data and data requests/responses between computer 112 and ventilator system 130. Alternatively, computer 112 may be directly connected to ventilator system 130 via serial or USB communication or via other suitable communication protocols.

Ventilator system 130 also includes a control unit 132, such as one or more processor devices, and a memory 134 accessible by control unit 132. Memory 134 includes logic, such as software or firmware, that contains instructions executable by control unit 132 for controlling operation of ventilator system 130. In one embodiment, memory 134 further includes one or more memory locations, such as cache memory locations, operative to temporarily store each updated set of the monitored patient data. Ventilator system 130 illustratively includes a local display 140 for displaying monitored data to medical professionals, the patient, and/or other individuals at the ventilator 130. Coupled to display 140 is a user interface 142 (e.g., graphical user interface, keyboard, mouse, etc.) providing a user with the ability to request, collect, and/or display particular data at ventilator system 130.

Ventilator system 130 includes a request/response mode of operation. In a request/response protocol, ventilator system 130 outputs available parametric data to an external device, such as computer 112, upon receipt of a data request from the external device, such as from computer 112. In one embodiment, ventilator system 130 collects more than 150 patient parameters following each respiration cycle, although any suitable number of patient parameters may be measured by ventilator system 130 depending on configuration. In one embodiment, the parameters are provided as a set to data collection system 100 upon request.

In one embodiment, ventilator system 130 is further configured to output a notification message automatically upon the completion of a respiration cycle by the monitored patient. An exemplary message includes a waveform representing the monitored air volume or pressure over time for the most recent respiration cycle. Other suitable messages may be provided, such as a data flag, etc. In the illustrated embodiment, this automatically generated message is output via port 156 and the parametric data is output (upon request) via port 154, although the message and parametric data may alternatively be output via the same port. In one operating mode of data collection system 100, data collection logic 150 uses the automatically generated message as a trigger to request updated parametric data from ventilator system 130, as described herein.

Data collection logic 150 (and data collection logic 50 of FIG. 1) is operative to sample data from ventilator system 130 based on a respiration rate of the patient monitored with ventilator system 130. In particular, data collection logic 150 dynamically adjusts the interval at which parametric data is sampled or polled from ventilator system 130 according to a monitored respiration or breathing rate of the monitored patient. In one embodiment, parametric data monitored by ventilator system 130 is subject to change or updates once every respiration cycle, i.e., inhalation/exhalation cycle. In other words, after an inhalation/exhalation cycle is complete, a new set of parametric data associated with the patient is available at ventilator system 130. In one embodiment, the cache of memory 134 is updated with each new set of parametric data following each respiration cycle. Thus, updated parametric data is available at ventilator system 130 at an asynchronous rate dependent on a patient's breathing rate. By adjusting the sampling rate to correspond with the detected respiration rate, the frequency of data requests from data collection logic 150 simulate the asynchronous availability of the parametric data. As such, data collection logic 150 grabs each or substantially each set of updated parametric data as it becomes available while reducing the likelihood of oversampling or under-sampling the parametric data. For example, oversampling includes sampling data more frequently than it changes at the ventilator system 130, and under-sampling includes sampling data less frequently than it changes at ventilator system 130.

While the data collection logic 150 is illustratively configured to sample data from ventilator system 130 following each exhalation phase of the respiration cycle, data collection logic 150 may alternatively sample data following each inhalation phase of the respiration cycle or at another suitable point in the respiration cycle of the patient.

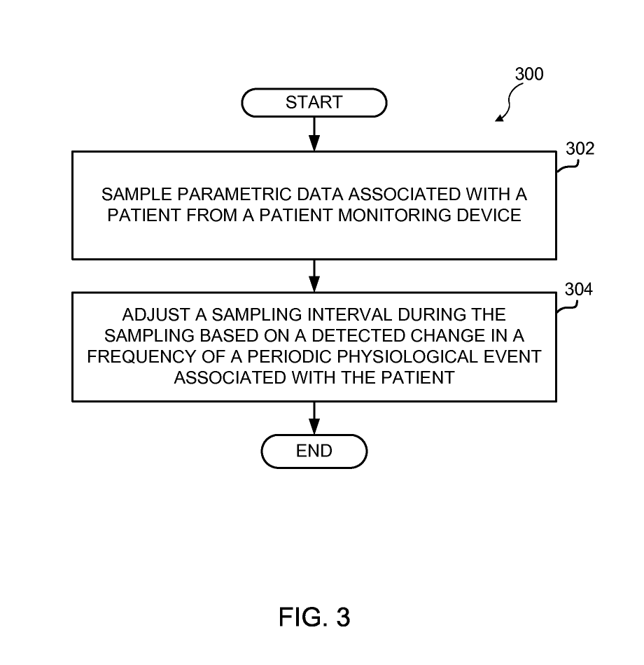

Referring to FIG. 3, a flowchart 300 of an exemplary method implemented by the data collection system 100 of FIG. 2 is illustrated. While FIG. 3 is described with respect to the data collection system 100 of FIG. 2, data collection system 10 of FIG. 1 is also operative to implement the method of FIG. 3. At block 302, data collection logic 150 samples parametric data associated with a monitored patient. The data, which is provided with the patient monitoring device (e.g., ventilator system 130), is sampled at the sampling frequency or rate implemented by logic 150. At block 304, data collection logic 150 adjusts the sampling rate during the sampling of the parametric data based on a detected change in a frequency of a periodic physiological event or response associated with the patient. For example, upon detecting a change in the respiration frequency of the patient monitored with ventilator system 130, data collection logic 150 adjusts the sampling rate based on the changed respiration frequency. As described herein, in one embodiment data collection logic 150 sets the sampling rate to match the detected respiration frequency. In another embodiment, data collection logic 150 sets the sampling rate such that data is sampled from ventilator system 130 upon receipt of a notification from ventilator system 130 that a respiration cycle has completed, as described herein.

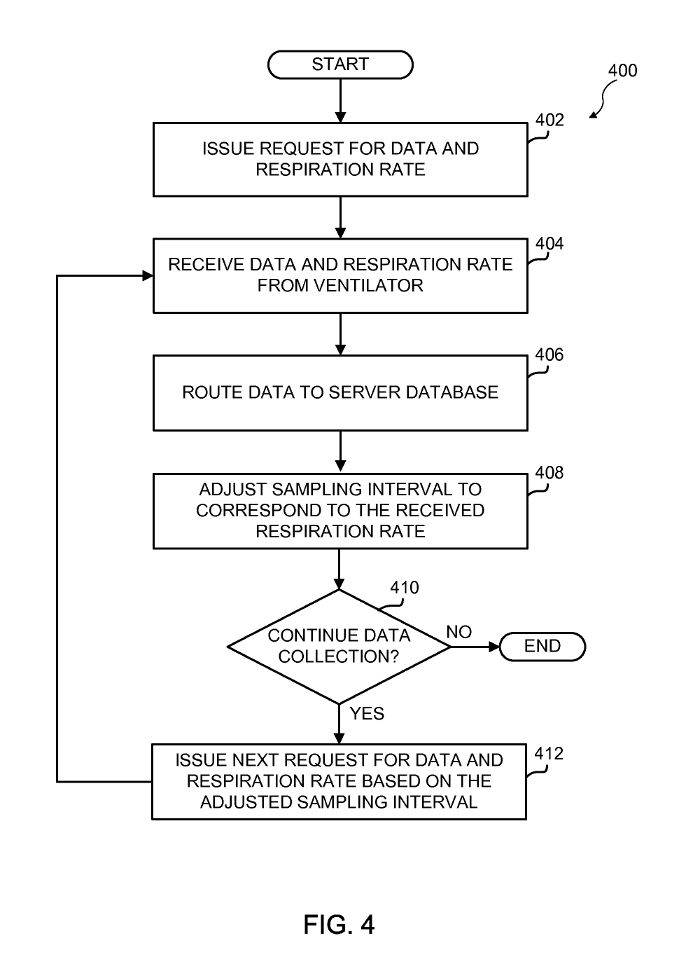

Referring to FIG. 4, a flowchart 400 of another exemplary method implemented by the data collection system 100 of FIG. 2 is illustrated. While FIG. 4 is described with respect to the data collection system 100 of FIG. 2, data collection system 10 of FIG. 1 is also operative to implement the method of FIG. 4.

At block 402, data collection system 100 initiates communication by sending a request to ventilator system 130 for the available parametric data at ventilator system 130. In addition, the request includes a request for an updated respiration rate of the monitored patient as determined by ventilator system 130. Ventilator system 130 determines the patient's respiration rate in any suitable fashion. For example, ventilator system 130 may include a sensor that monitors the patient's respiration. The sensor may detect air pressure or volume, for example, to determine the completion of each respiration cycle, and thus to determine the respiration rate. Alternatively, a respiration rate that is input by an operator (e.g., clinician, nurse, etc.) may be used as the actual respiration rate that is communicated to data collection system 100. For example, ventilator system 130 may include a control mode where the respiration rate of the patient is controlled at a specified, fixed rate as entered by the operator. In one embodiment, ventilator system 130 continually monitors the respiration rate, i.e., updates the respiration rate following each respiration cycle.

Ventilator system 130 outputs (e.g., via port 154) the most recent respiration rate information and parametric data upon receipt of the request at block 402. Computer 12 receives the respiration rate information and parametric data set from ventilator system 130 at block 404, illustratively via link 162 at communication port 158 of FIG. 2.

Alternatively, ventilator system 130 provides information related to the patient's respiration rate to computer 12 at block 404, and computer 12 determines the updated respiration rate of the patient based on the received respiration information. For example, ventilator system 130 may provide data related to the detected air pressure and/or volume following the respiration cycle of the patient. Based on the received data, computer 12 calculates the updated respiration rate.

At block 406, data collection logic 150 routes the received parametric data to database 114 for storage. In addition, based on the updated respiration rate, data collection logic 150 adjusts the sampling interval at block 408 such that the frequency at which system 100 collects data, i.e., the frequency at which system 100 issues data requests and receives data, corresponds to or matches the respiration rate received at block 404. For example, if the monitored patient is breathing at a respiration rate of ten breaths per minute, data collection logic 150 sets the sampling interval to six seconds, i.e., a sampling frequency of ten samples per minute.

Upon continued data collection at block 410, data collection logic 150 issues the next request for parametric data and respiration rate at block 412 based on the sampling interval determined at block 408. Since the next request at block 412 is based on the adjusted sampling interval, the request is configured to collect the next available parametric data and respiration rate at ventilator system 130. Data collection logic 150 then proceeds to block 404 to receive the updated data and respiration rate and to continue the data collection and adjustment of the sampling rate during the data collection. Because the sampling interval determined at block 408 corresponds to the updated respiration rate of the patient, each successive request issued at block 412 is operative to grab the updated data set and respiration rate from ventilator system 130 following each respiration cycle of the patient.

In an exemplary data collection sequence, data collection system 100 samples data at an interval of six seconds corresponding to a respiration rate of ten respiration cycles per minute. As such, a next request (issued at block 412) is issued six seconds after a previous request. The updated respiration rate received from ventilator system 130 following the next request is 12 respiration cycles per minute, for example. As such, data collection logic 150 updates the sampling frequency at block 408 to 12 samples per minute, and the following request is issued 5 seconds after the previous request. Thus, the sampling rate is continually updated with each successive breathing cycle of the patient until the data collection stops at block 410.

In one embodiment, adjusting the sampling rate based on each received respiration rate (with each respiration cycle) increases the likelihood of maintaining a sampling rate that matches the patient's actual respiration rate, and thus increases the likelihood of acquiring all updated parametric data associated with each respiration cycle of the patient. However, in some embodiments, a less frequent sampling rate may be implemented. For example, in another embodiment, data collection system 100 is configured to adjust the sampling rate less frequently, such as every other breathing cycle, every 10 seconds, etc.

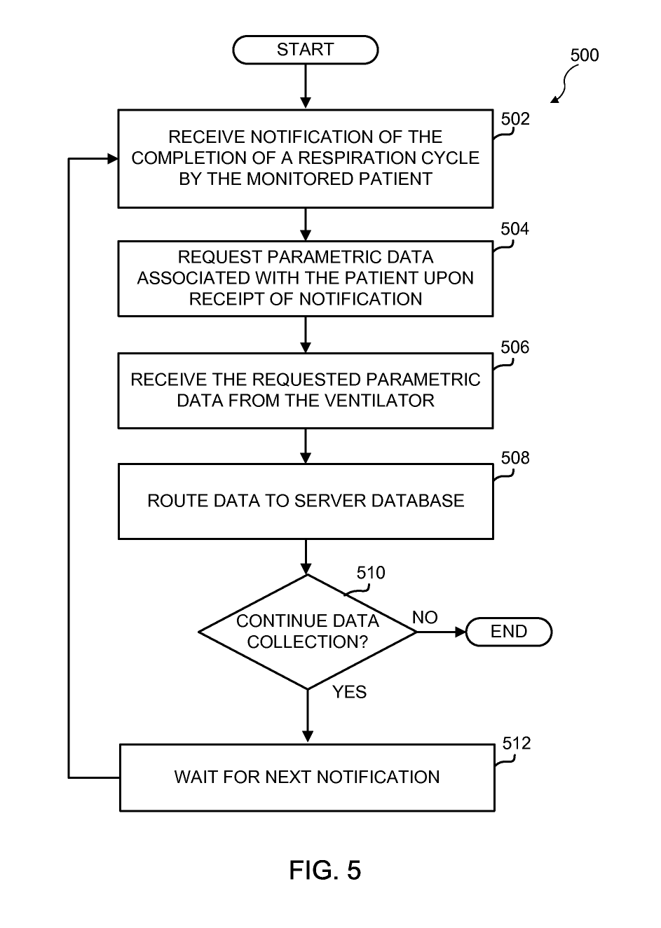

Referring to FIG. 5, a flowchart 500 of another exemplary method implemented by the data collection system 100 of FIG. 2 is illustrated. The method of FIG. 5 incorporates the use of the notification message automatically generated by ventilator system 130 (described herein) to adjust the sampling rate. In one embodiment, the operation of FIG. 5 is performed upon configuring the ventilator system 130 into a mode such that it automatically generates the notification message and upon configuring data collection system 100 to sample data based on the notification message. While FIG. 5 is described with respect to the data collection system 100 of FIG. 2, data collection system 10 of FIG. 1 is also operative to implement the method of FIG. 5.

At block 502, upon connection of data collection system 100 to ventilator system 130, data collection system 100 receives a notification from ventilator system 130 upon completion of a respiration cycle by the monitored patient. In the illustrated embodiment, the notification received at block 502 is the data message automatically provided by ventilator system 130 via port 156, as described herein. In one embodiment, the message is a waveform representing the monitored air volume or pressure over time for the most recent respiration cycle. However, the message may include any suitable signal or data flag that is configured to automatically generate upon completion of a breathing cycle by the patient. The message serves as a notification to data collection system 100 that there is updated parametric data available at ventilator system 130 corresponding to the completion of the breathing cycle. As such, the receipt of the message at data collection system 100 serves as a trigger for data collection logic 150 to initiate a data request, as represented at block 504.

Upon sending a request to ventilator system 130 at block 504 following receipt of the notification message at block 502, data collection system 100 receives the updated data set from ventilator system 130 at block 506. In one embodiment, data collection system 100 also receives the respiration rate, as described herein with respect to FIG. 4, but does not adjust the sampling rate based on the respiration rate. At block 508, data collection logic 150 routes the received data to database 114 for storage. If data collection is continued at block 510, data collection logic 150 waits for the next notification from ventilator system 130 that would indicate the completion of the next successive breathing cycle of the patient before issuing the next data request, as represented at block 512. As such, the flow diagram returns to block 502 upon receipt of the next notification message, and data collection logic 150 issues the next data request at block 504. As such, the sampling rate implemented by data collection logic 150 in the operational mode of FIG. 5 is directly tied to the rate at which the notification messages are received from ventilator system 130. The data collection continues until the operation is stopped at block 510.

In one embodiment, data collection logic 150 implements a maximum and/or a minimum sampling rate in the methods of FIGS. 3-5 to define boundaries within which the adjusted sampling rate is maintained. For example, if a patient is hyperventilating or breathing at a fast rate, sampling data at the same rate as the respiration rate may consume considerable bandwidth and overburden the ventilator system 130. As such, if the adjusted sampling rate determined by computer 112 exceeds a maximum threshold rate, the sampling rate implemented by data collection logic 150 is held at the maximum threshold rate. An exemplary maximum threshold rate is twenty samples per minute, i.e., a sampling interval of three seconds. Other suitable maximum sampling rates may be implemented depending on the design capabilities of ventilator system 130 and data collection system 100 as well as the available bandwidth and data speed of the communication link between the systems 100, 130. In addition, a minimum sampling rate may also be implemented by data collection logic 150 such that data is requested by system 100 at least as frequently as the minimum sampling rate.

When data collection system 100 simultaneously collects data from multiple patient monitoring devices 30, data collection logic 150 is operative to set a sampling rate for each device 30 based on the detected respiration rate of the patient connected to each device 30. As such, data collection system 100 may sample data provided with each of a plurality of patient monitoring devices 30 at a different sampling frequency.

While the methods of FIGS. 4 and 5 are described with respect to data collection from a ventilator system 130, the methods of FIGS. 4 and 5 may be implemented to collect data from a heart monitor system, a pulse oximetry device, or any suitable patient monitoring device that is operative to detect a periodic physiological event or response of a patient. For example, data collection systems 10, 100 of FIGS. 1 and 2 may be used to collect data from a heart monitor system or from a pulse oximetry device. Rather than adjusting the sampling rate based on the respiration rate (as with a ventilator), the data sampling rate is adjusted based on the detected heart rate or pulse of the monitored patient. As such, in the operation of FIG. 4, the heart monitor system or pulse oximetry device provides a pulse rate to data collection system 100 following each data sampling request, and data collection logic 150 adjusts the sampling rate in accordance with each received pulse rate. Similarly, in the operation of FIG. 5, the heart monitor system or pulse oximetry device generates a notification message automatically upon the patient's completion of a pulse cycle, and data collection logic 150 issues a data request based upon the receipt of the notification message. Alternatively, data collection system 100 may adjust the sampling rate and collect data less frequently than with each pulse, such as, for example, every other detected pulse, every third pulse, etc. In another example, an accelerometer or other suitable sensing device is used to detect a posture change of the patient. Upon detection of a posture change with the accelerometer, data collection system 100 samples parametric data, such as blood pressure or other suitable data, associated with the monitored patient. Other suitable patient monitoring systems that monitor a periodic physiological event or response of a patient may be used with the system and method of the present disclosure.

Although the invention has been described in detail with reference to certain preferred embodiments, variations and modifications exist within the spirit and scope of the invention as described and defined in the following claims.

1. A method of sampling parametric data by a data collection system, the parametric data being provided with a ventilator system and being associated with a patient monitored by the ventilator system, the method including:
receiving information associated with a first respiration rate of a monitored patient, the information associated with the first respiration rate being provided with the ventilator system;
setting a data sampling rate to substantially match the first respiration rate;
sampling parametric data associated with the monitored patient at the data sampling rate, the parametric data being provided with the ventilator system;
receiving information associated with a second respiration rate of the monitored patient, the second respiration rate being different from the first respiration rate;
adjusting, during the sampling of the parametric data, the data sampling rate to substantially match the second respiration rate; and
sampling parametric data associated with the monitored patient at the adjusted data sampling rate.

2. The method of claim 1, further including sending a request for the parametric data associated with the patient to the ventilator system.

3. The method of claim 2, further including receiving the requested parametric data and a respiration rate from the ventilator system upon sending the request.

4. The method of claim 1, further including storing the received parametric data in a server database of the data collection system.

5. The method of claim 1, further including receiving a notification from the ventilator system following each of a plurality of successive respiration cycles of the monitored patient, the notification indicating that parametric data associated with the previous respiration cycle of the patient is available at the ventilator system.

6. The method of claim 5, further including:
sending a request to the ventilator system for the available parametric data associated with the previous respiration cycle of the patient following the receipt of each notification; and
receiving the requested parametric data at the data collection system following the sending of each request.

7. The method of claim 1, further including limiting the adjusted data sampling rate to a predetermined maximum rate upon the second respiration rate exceeding the predetermined maximum rate.

8. The method of claim 1, wherein the first and second respiration rates are received following a request from the data collection system to the ventilator system for parametric data.

9. The method of claim 1, further including determining the first respiration rate based on the received information associated with the first respiration rate, and determining the second respiration rate based on the received information associated with the second respiration rate.

10. A method of sampling data provided with a patient monitoring device, the method including:
sampling parametric data associated with a patient and provided with a patient monitoring device;
determining a frequency of a periodic physiological event associated with the patient; and
adjusting a sampling rate during the sampling of the parametric data based on a change in the frequency of the periodic physiological event associated with the patient.

11. The method of claim 10, further including receiving information associated with the frequency of the periodic physiological event from the patient monitoring device.

12. The method of claim 11, further including setting the sampling rate to match the frequency of the periodic physiological event associated with the patient.

13. The method of claim 12, further including limiting the sampling rate to a predetermined maximum rate upon the frequency of the periodic physiological event exceeding the predetermined maximum rate.

14. The method of claim 10, further including receiving a notification of each of a plurality of successive occurrences of the periodic physiological event associated with the patient.

15. The method of claim 14, wherein the sampling rate is adjusted to sample the parametric data associated with the patient following the receipt of each notification.

16. The method of claim 10, wherein the periodic physiological event associated with the patient includes at least one of respiration and pulse.

17. The method of claim 10, further including sampling parametric data associated with the monitored patient at the adjusted sampling rate, and storing the sampled parametric data in a server database.

18. A data collection system including:
a database; and
at least one server computer operative to sample parametric data associated with a patient and to store the sampled parametric data in the database, the parametric data being provided with a patient monitoring device, the at least one server computer adjusting a sampling frequency during a sampling of the parametric data based on a change in a determined frequency of a periodic physiological event associated with the patient.

19. The data collection system of claim 18, wherein the at least one server computer is operative to sample parametric data provided with each of a plurality of patient monitoring devices at a different sampling frequency.

20. The data collection system of claim 18, wherein the at least one server computer receives information associated with the frequency of the periodic physiological event from the patient monitoring device.

21. The data collection system of claim 20, wherein the at least one server computer is further operative to set the sampling frequency to match the frequency of the periodic physiological event associated with the patient.

22. The data collection system of claim 18, wherein the at least one server computer is further operative to receive a notification of each of a plurality of successive occurrences of the periodic physiological event associated with the patient from the patient monitoring device.

23. The data collection system of claim 22, wherein the at least one server computer is further operative to sample the parametric data associated with the patient following the receipt of each notification.

24. The data collection system of claim 18, wherein the periodic physiological event associated with the patient includes at least one of respiration and pulse.
